(19) United States
(12) Patent Application Publication
(10) Pub. No.: US 2021/0100997 A1
(43) Pub. Date: Apr. 8, 2021

(54) ANTI-INFECTIVE PROTECTIVE DEVICE

(71) Applicant: Lorie Rangel, Delano, CA (US)

(72) Inventor: Lorie Rangel, Delano, CA (US)

(21) Appl. No.: 16/591,768

(22) Filed: Oct. 3, 2019

(51) Int. Cl. A61M 39/20; A61L 29/16; A61M 39/02

(52) U.S. Cl. CPC A61M 39/20; A61L 2300/404; A61M 39/0247; A61L 29/16

(57) ABSTRACT

An anti-infective protective device including a cap member and a connector ring member. The cap member includes an enclosed pad having a disinfectant solution immersed therein, which is sealed in the cap member by a covering attached to the cap member. The cap member disinfects and covers a received port attached which is attached to a tubing element. The connector ring member is attached to at least one tubing element at one end and is attached to the cap member at an opposite end. The connector ring member integrates the cap member with a structure of the tubing element.

### FIELD OF THE INVENTION

Embodiments described herein generally relate to protective devices, and more particularly to an anti-infective protective device.

### BACKGROUND OF THE INVENTION

The use of medical tubing in hospitals absent the necessary precaution can lead to disease and infection. The tip of the medical tubing set can be exposed to potential contaminants, which can lead to infection if the non-sterile medical tubing set is reconnected to a patient's IV access. Moreover, the ports attached to the medical tubing may be exposed to potential contaminants that can be pushed into the patient's IV line after the port has been accessed by tubing or a syringe. This is problematic for both the person seeking care and for the medical practitioners tasked with the recovery of the patient. Hence, it is desirable to provide an effective means for safeguarding against the contraction of a possible infection from the use of medical tubing.

### BRIEF DESCRIPTION OF THE DRAWINGS

The various advantages of the embodiments of the present disclosure will become apparent to one skilled in the art by reading the following specification and appended claims, and by referencing the following drawing(s), in which:

FIG. 1 shows an exemplary view of an anti-infective protective device according to an embodiment of the present disclosure.

FIG. 2 shows an exemplary view of the anti-infective protective device connected to at least one tubing element according to an embodiment of the present disclosure.

### SUMMARY OF THE INVENTION

Exemplary embodiments disclosed herein describe an anti-infective protective device including at least one cap member and at least one connector ring member. The cap member includes an enclosed pad having a disinfectant solution immersed therein, which is sealed in the cap member by a covering attached to the cap member. The cap member disinfects and covers a received port attached which is attached to a tubing element. The connector ring member is attached to at least one tubing element at one end and is attached to the cap member at an opposite end. The connector ring member integrates the cap member with a structure of the tubing element.

In some exemplary embodiments, each tubing element includes one or more port sites where a port is connected to the tubing element.

In some exemplary embodiments, a connector ring member is attached to each tubing element at each port site attached along the tubing element.

In some exemplary embodiments, a cap member is attached to each connector ring member which is attached to each tubing element at each port site along the tubing element.

In some exemplary embodiments, the covering attached to the cap member is removed and contents of the enclosed pad are released upon the received port to disinfect the received port when the cap member is attached to the received port.

In some exemplary embodiments, the at least one connector ring member includes a rubber strip having a rubber circular attachment element at one end of the strip.

In some exemplary embodiments, the rubber circular attachment element attaches to the at least one tubing member.

In some exemplary embodiments, the cap member is attached directly to the other end of the rubber strip.

In some exemplary embodiments, the at least one tubing element is a medical tubing element.

In some exemplary embodiments, the at least one tubing element is an intravenous (IV) tubing element.

In some exemplary embodiments, the at least one tubing element is an extension tubing element.

In some exemplary embodiments, the disinfectant solution is chlorohexidine.

### DETAILED DESCRIPTION

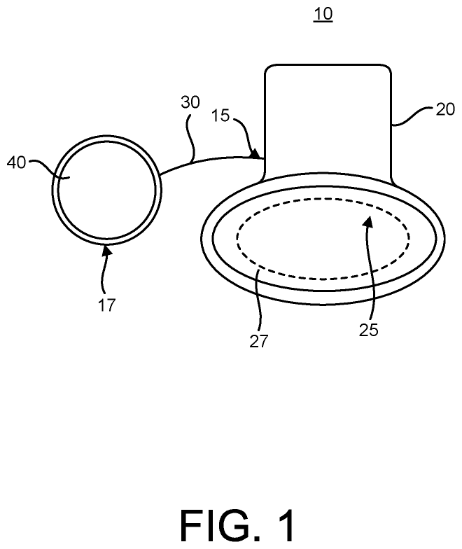

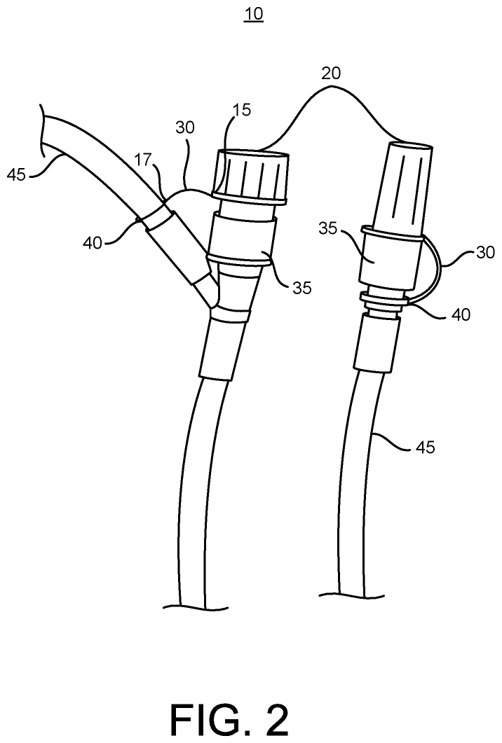

The present disclosure relates to an anti-infective protective device (“the device”). The device includes a safeguard cap enclosed with a disinfectant, which is sealed within the device by a thin plastic film, and a connector ring which connects the safeguard cap to a tubing component The device effectively provides a built-in protective covering including a disinfectant for tubing components to protect against possible infection which may be contracted from certain types of tubing components. As shown in FIGS. 1-2, an exemplary device 10 may include at least one cap member 20 and at least one connector ring member 30, 40. The device 10 may be made from any suitable material. In a preferred embodiment, the device is made of rubber.

The cap member 20 may be fixedly attached to the connector ring member 30, 40 at a proximate end 15. The cap member 20 may be impregnated (i.e., enclosed) with a pad 25, which may be immersed in a disinfectant solution, such as, for example, chlorohexidine. The impregnated pad may be sealed within the cap member by a thin protective film 27, such as, for example, a plastic film. The cap member may be configured to receive a port 35 which is attached to a tubing element 45, such as, for example, a medical tubing element. The port is attached to the tubing element at a port site. The cap member 30 may have any suitable shape, such as, for example, circular or triangular.

The connector ring member 30, 40 may include strip 30 which may be fixedly connected to circular attachment element 40 (i.e., a rubber ring). The circular attachment element may be attachably connected to tubing element 45. As noted, cap member 20 may be fixedly attached to the connector ring member. As such, the connector ring member may be fixedly attached to cap member 20 at its proximate end 15 and may be attachably connected to tubing element 45 at its distal end 17, which thereby integrates the cap member 20 with the structure of the tubing element.

In one aspect, a port 35 may be an access port of a medical tubing 45 element, such as, for example, an intravenous (IV) tubing element (e.g., primary lines, secondary lines and extension lines). A medical tubing element may include one port site (i.e., location where a port is attached to the tubing element) or multiple port sites along the medical tubing element. A port may be used as an injection port for receiving fluids which are to be transmitted to an individual (e.g., a patient) through the individual's access port. The injection ports and the patient's access port may be exposed to potential contaminants if the ports are not covered. As such, cap member 20 and connector ring member 30, 40 may be attached to each port 35 attached to a medical tubing element 45 to disinfect the port and to provide a safeguard covering for the port.

In another aspect, multiple tubing elements may be used, and each tubing element may include one port site or multiple port sites along the tubing element. In this aspect, the connector ring member 30, 40 and cap member 20 are attached to each tubing element 45 at each port site along the tubing element.

When cap member 20 is attached to a port 35, the protective film 27 is removed from the cap member. Next, squeezing friction is applied to the cap member to release the disinfectant solution immersed in the enclosed pad 25. The released disinfectant solution is immersed onto the port 35 to disinfect (i.e., sterilize) the port when the port is inserted into the cap member. The cap member may attach to the port by any suitable mechanism, such as, for example, screwing onto a thread of the port. The cap member may remain attached to the port until subsequent use of the port is needed, thereby providing a safeguard covering to the attached port.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.)

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

What is claimed is:

1. An anti-infective protective device comprising:
at least one cap member including an enclosed pad having a disinfectant solution immersed therein, the enclosed pad is sealed within the cap member by a covering attached to the cap member, wherein the cap member disinfects and provides a safeguard covering for a received port attached to a tubing element; and
at least one connector ring member attached to at least one tubing element at one end and attached to the cap member at an opposite end, wherein the connector ring member integrates the cap member with a structure of the tubing element.

2. The device of claim 1, wherein each tubing element includes one or more port sites where a port is connected to the tubing element.

3. The device of claim 2, wherein a connector ring member is attached to each tubing element at each port site attached along the tubing element.

4. The device of claim 3, wherein a cap member is attached to each connector ring member which is attached to each tubing element at each port site along the tubing element.

5. The device of claim 1, wherein the covering attached to the cap member is removed and contents of the enclosed pad are released upon the received port to disinfect the received port when the cap member is attached thereto.

6. The device of claim 1, wherein the at least one connector ring member includes a rubber strip having a rubber circular attachment element at one end of the strip.

7. The device of claim 6, wherein the rubber circular attachment element attaches to the at least one tubing member.

8. The device of claim 7, wherein the cap member is attached directly to the other end of the rubber strip.

9. The device of claim 1, wherein the at least one tubing element is a medical tubing element.

10. The device of claim 1, wherein the at least one tubing element is an intravenous (IV) tubing element.

11. The device of claim 1, wherein the at least one tubing element is an extension tubing element.

12. The device of claim 1, wherein the disinfectant solution is chlorohexidine.
